(19) United States
(12) Patent Application Publication
(10) Pub. No.: US 2023/0273232 A1
(43) Pub. Date: Aug. 31, 2023

(54) ROBOTIC MICROINJECTION

(71) Applicant: RoboBio, LLC, Sacramento, CA (US)

(72) Inventors: Grant J. Gallagher, Irvine, CA (US); Mark J. Gallagher, Irvine, CA (US)

(21) Appl. No.: 17/976,677

(22) Filed: Oct. 28, 2022

Related U.S. Application Data
(60) Provisional application No. 63/314,302, filed on Feb. 25, 2022.

(51) Int. Cl. G01N 35/10; G01N 35/00

(52) U.S. Cl. CPC G01N 35/1011; G01N 35/00732; G01N 35/0099; G01N 2035/1039

(57) ABSTRACT

Apparatus and methods for more efficiently micro-positioning a micropipette with respect to an egg and/or injecting fluid into a plurality of eggs using a micropipette are provided. Various approaches described herein comprise use of automated and/or robotic systems to direct a micropipette to and inject the micropipette in eggs mounted on a sample holder.

### PRIORITY CLAIM

This application claims the priority benefit of U.S. Patent Prov. App. 63/314,302, titled ROBOTIC MICROINJECTION, filed Feb. 25, 2022, which is hereby incorporated herein by reference in its entirety.

### BACKGROUND OF THE INVENTION

### Field

The present disclosure relates to robotic microinjection of eggs and embryos such as insect eggs and embryos.

### Description of the Related Art

Malaria continues to impose hardship on much of the world causing illness and death. As is well known, malaria is transmitted by mosquitoes and regions with climates conducive to producing large mosquito populations encounter higher rates of malaria.

A variety of measures are being undertaken to alleviate the disease ranging from efforts to reduce standing water and use of mosquito nets to larvicides and insecticides. Malaria vaccines are even being developed. Use of transgenic mosquitoes offers a potentially promising approach to vector control. Transgenic mosquitoes are mosquitoes that are genetically modified, for example, by injecting a particularly selected gene into mosquito eggs using a pipette. Transgenic mosquitoes, for example, have been produced that cause female offspring of the transgenic mosquitoes to die before reaching full maturity. Such transgenic mosquitoes have been released in locations in the Brazil, Cayman Islands, Panama, India and United States. Transgenic mosquito solutions are directed to specific target species of mosquitoes. Research continues in developing transgenic mosquitoes for mosquito vector control. Such solutions may help reduce the incidence of malaria as well as other mosquito borne diseases such as Dengue fever, West Nile virus, and Zika and yellow fever. Techniques for improving and expediting research endeavors in genetical modified mosquitoes may thus be beneficial.

### SUMMARY

The present disclosure relates generally to apparatus and methods for more efficiently micro-positioning a micropipette with respect to an egg and/or injecting fluid into a plurality of eggs using a micropipette. Various approaches described herein comprise use of automated and/or robotic systems to direct a micropipette to and inject the micropipette in eggs mounted on a sample holder. For example, various designs disclosed herein comprise a micro-positioning system configured to insert a pipette into a plurality of eggs, wherein the micro-positioning system comprises:
a pipette holder configured to secure the pipette to the pipette holder;
a shaft for mounting a plurality of eggs, the pipette holder movable in lateral, longitudinal, and vertical directions with respect to the shaft to position the pipette with respect to the eggs;
at least one electronically controlled lateral translation stage configured to translate in the lateral direction so as to move the pipette holder laterally with respect to the shaft;
at least one electronically controlled longitudinal translation stage configured to translate in the longitudinal direction so as to move the pipette holder longitudinally with respect to the shaft;
at least one vertical translation stage configured to translate in the vertical direction so as to move the pipette holder vertically with respect to the shaft;
a motor connected to said shaft to rotate said shaft so as to provide the pipette access to different eggs mounted on the shaft; and
electronics configured to drive at least the lateral and longitudinal translation stages and the motor to position the pipette with respect to the egg.

Some designs disclosed herein comprise a micro-positioning system configured to insert a pipette into a plurality of elongate eggs, the elongate eggs having ends separated by a length and having sides separated by a wide, the length longer than the width, wherein the micro-positioning system comprises:
a pipette holder configured to secure the pipette to the pipette holder;
a platform for mounting a plurality of elongate eggs, the pipette holder movable in lateral, longitudinal, and vertical directions with respect to the platform to position the pipette with respect to the elongate eggs;
at least one electronically controlled lateral translation stage configured to translate in the lateral direction so as to move the pipette holder laterally with respect to the platform;
at least one electronically controlled longitudinal translation stage configured to translate in the longitudinal direction so as to move the pipette holder longitudinally with respect to the platform;
at least one vertical translation stage configured to translate in the vertical direction so as to move the pipette holder vertically with respect to the platform;
at least one camera including microscope optics configured to images the pipette and the elongate eggs, and
electronics configured to drive at least the lateral and longitudinal translation stages to position the pipette with respect to the elongate egg, the electronics configured to received images from the at least one camera of the eggs and the pipette and to drive at least the lateral and longitudinal translation stages to position the pipette with respect to the end of the egg such that the pipette penetrates the elongate egg based on the images from the camera.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 is a top view of pipette secured to a pipette holder mounted on a laterally translating translation stage and a longitudinal translating translation stage for moving the pipette in the x and z directions, respectively. FIG. 1 also shows a plurality of eggs arranged in a row along the edge of a microscope slide for injection by the pipette.

FIG. 2 is a cross-sectional view of the pipette on the x and z translation stages and an egg on the microscope slide showing the pipette position to penetrate the egg with forward movement of the z translation stage.

FIG. 3 is a side cross-sectional view of the pipette on the x and z translation stages and a plurality of eggs on the microscope slide showing the pipette position to penetrate an egg on the edge of the microscope slide. In the configuration shown, this egg located at the edge of the microscope slide blocks access of the pipette to the other two eggs shown.

FIG. 4 is a perspective view of pipette secured to a pipette holder mounted on x, y, and z translations stages configured to move the pipette in the x, y and z directions, respectively, to eggs on a rotatable shaft connected to a motor configured to rotate the shaft to provide access to eggs located at different positions about the circumference of the shaft. One or more of these translations stages as well as the motor may be electrically controlled.

FIG. 5 is a side cross-sectional view of the eggs on the rotating shaft and the pipette positioned to penetrate the end of the egg on the top of the shaft. The shaft can rotate to move different eggs into position on the top of the shaft in proximity to the pipette to provide access to the pipette to the egg. A microscope camera above the shaft is configured to images the pipette and the eggs, in particular, the eggs on the top of the shaft.

FIG. 6 is a top view of the shaft showing a plurality of eggs thereon. The pipette can be laterally and longitudinally translated (in x and y directions, respectively) to contact the ends of eggs within a zone, e.g., at the top of the shaft. The shaft can be rotated to position other eggs on the shaft in that zone.

FIG. 7 is a perspective view of the shaft connected to a motor and having eggs thereon mounted on a rotation stage such that the shaft can be rotated about an axis orthogonal to the length of the shaft.

FIG. 8 is a top view of the shaft showing how the shaft can be rotated by the rotation stage shown in FIG. 7 to re-orient the eggs such that the pipette can be injected into the end of the elongate eggs.

FIG. 9 is a block diagram showing a microprocessor electrically connected to top and side cameras as well as drivers for x, y, and z electronically controlled translation stages that are electrically connected to the respective translation stages. The microprocessor is also shown electrically connected to a stepper motor driver that is electrically connected to the motor for rotating the shaft as well as a driver for driving the rotation stage.

FIG. 10 is a flow chart illustrating an example method for penetrating eggs mounted on the rotating shaft with the pipette based on images of the pipette and eggs obtained from a camera.

### DETAILED DESCRIPTION OF CERTAIN INVENTIVE ASPECTS

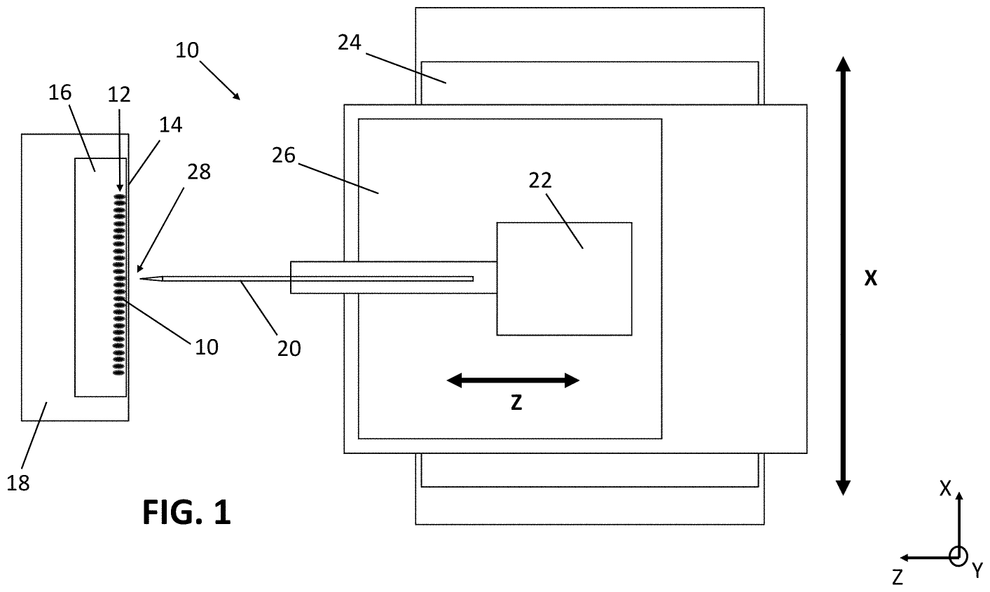

One method of microinjection into eggs 10 such as mosquito eggs is to line the eggs up in a row 12 along the edge 14 of a microscope slide 16 as illustrated in FIG. 1. The microscope slide 16 is shown on a platform 18 for support. FIG. 1 also shows a pipette 20 secured to a pipette holder 22 that is mounted on first and second translations stages 24, 26. The first and second translations stage 24, 26 may be configured to move in orthogonal directions. The first and second translation stages 24, 26 may comprise, for example, a lateral translation stage and a longitudinal translation stage. The lateral translation stage 24 may, for example, be configured to translate in a lateral direction such as in the x direction shown in FIG. 1 (e.g., parallel to the x axis), possibly in a direction left or right with respect to the eggs 10. The longitudinal translation stage 26 may, for example, be configured to translate in a longitudinal direction such as in the z direction shown in FIG. 1 (e.g., parallel to the z axis) in a direction toward the eggs 10. A third translation stage (not shown) may be configured to translate in a direction orthogonal to the first and second translation stages 24, 26. This third translation stage may, for example, be configured to translate in the vertical direction (e.g., parallel to the y axis), possibly in a direction up or down with respect to the eggs 10. Accordingly, the lateral and vertical translation stages may be employed to align the pipette 20, and in particular, a tip 28 of the pipette with eggs 10 on the edge 14 of the microscope slide 16. The longitudinal translation stage 26 can translate the pipette 20 forward so that the tip 28 of the pipette penetrates the egg 10 for injection.

Other configurations are possible. For example, the eggs 10 need on be on a microscope slide 16. Instead of the microscope slide 16, a microscope slip could be used. Moreover, in various implementations, the eggs 10 are supported by a platform 18 other than a microscope slide 16 or microscope slip. Also, although two translation stages 24, 26 are shown, more translation stages may be included. Additionally, although the translation stages 24, 26 are shown mounted to the pipette holder 22 and pipette 20, one or more of the translation stages may be mounted to the sample platform 18. Furthermore, both the sample platform 18 and the pipette holder 22 may be mounted to translations stages and the translation stage(s) mounted to the platform may move along same or different directions as the translation stage(s) mounted to and configured to move the pipette holder and pipette 20.

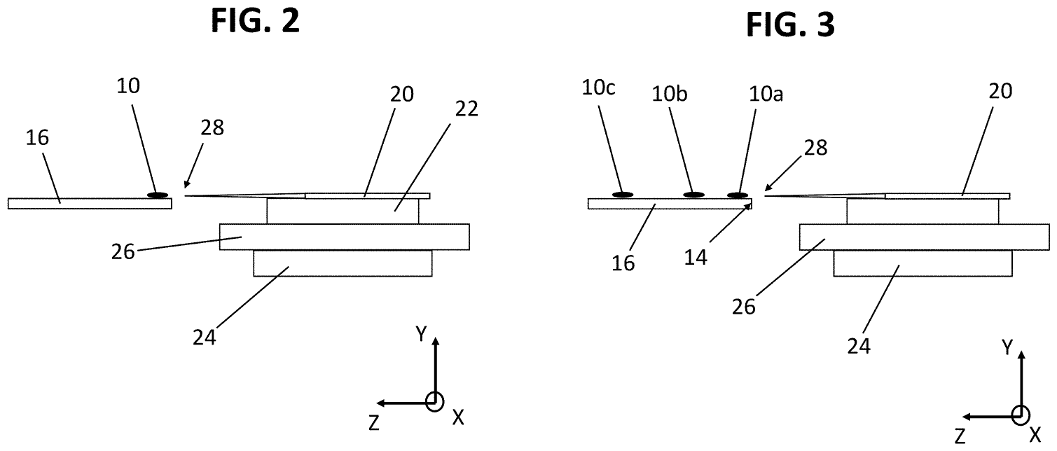

FIG. 2 is a cross-sectional view of the pipette 20 on the x and z translation stages 24, 26 and an egg 10 on the microscope slide 16 showing the pipette positioned to penetrate the egg with forward movement of the z translation stage. The egg 10 is elongate with first and second ends separated by a length and sides separated by a width. In this example, the first end of the egg is closer the pipette 20, pipette tip 28 and pipette holder 22 than the second opposite end. Also, in this case the length of the egg 10 is oriented along the longitudinal direction (e.g., parallel to the z-axis). The first end of the egg 10 is closer to the pipette 20. The pipette holder 20 and pipette 20 secured thereto are at a height such that the tip 28 of the pipette is at the same height as the center of the end of the egg 10 closest to the pipette tip. Although not shown, the lateral position of the pipette tip 28, e.g., the position of the pipette tip 28 along the lateral direction, which in this example is shown parallel to the x axis, is the same as the lateral position of the first end of the egg. With the height and lateral position of the pipette tip 28 aligned with that of the first end of the egg 10, the pipette 20 is positioned so as to penetrate the egg with movement of the pipette 20 in the longitudinal direction, e.g., in a direction parallel to the z-axis. The longitudinal translation stage 26 can thus move the pipette holder 22 and the pipette 20 such that the pipette tip 28 contact the first end of the egg and proceed into the egg.

As shown in FIG. 3, if additional eggs 10b, 10c are included on the microscope slide 16 not on the egg 14 or proximal to the edge of the microscope slide, the egg(s) 10a on the edge of the microscope slide may block access of the pipette to the egg(s) further in from the edge. Accordingly, as shown in FIG. 1, the eggs 10 are arranged in a row 12. At least in the example shown in FIG. 1, the pipette 20 is not configured to access additional eggs 10 not in the row 12 of eggs along the edge 14 of the microscope slide 16. In theory, the row 12 eggs 10 need not be located at the edge 14 of the microscope slide 16 if the slide is oriented level with respect to the path of the pipette 20 in the longitudinal direction e.g., with respect to the z-axis. In any case, however, if the slide 16 is oriented level with respect to the path of the pipette 20 in the longitudinal direction e.g., with respect to the z-axis, one egg 10 may block the pipette from reaching another egg that is in the way of the longitudinally translating pipette.

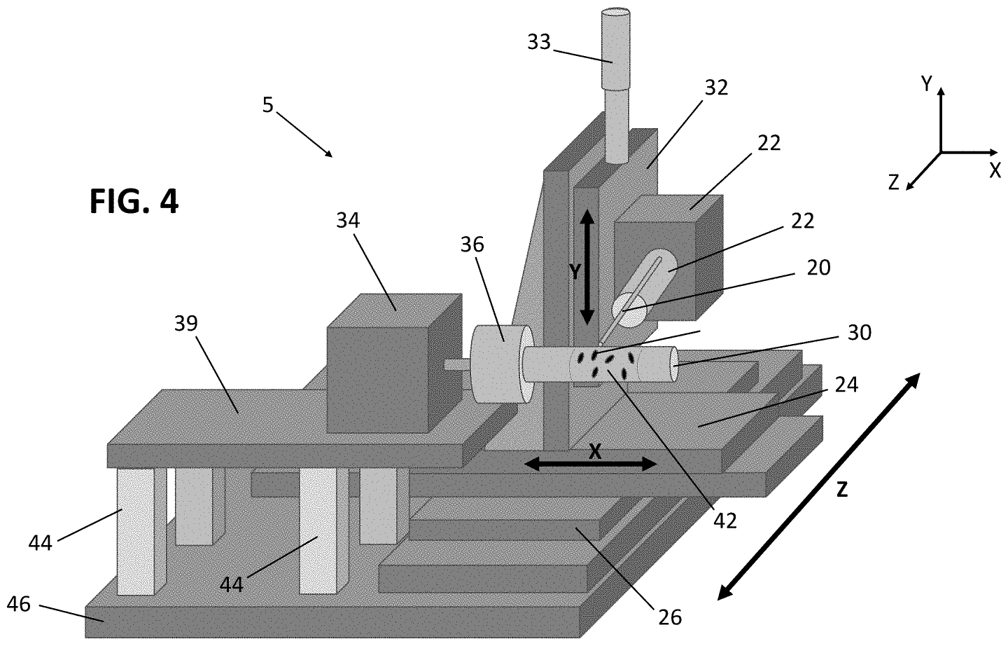

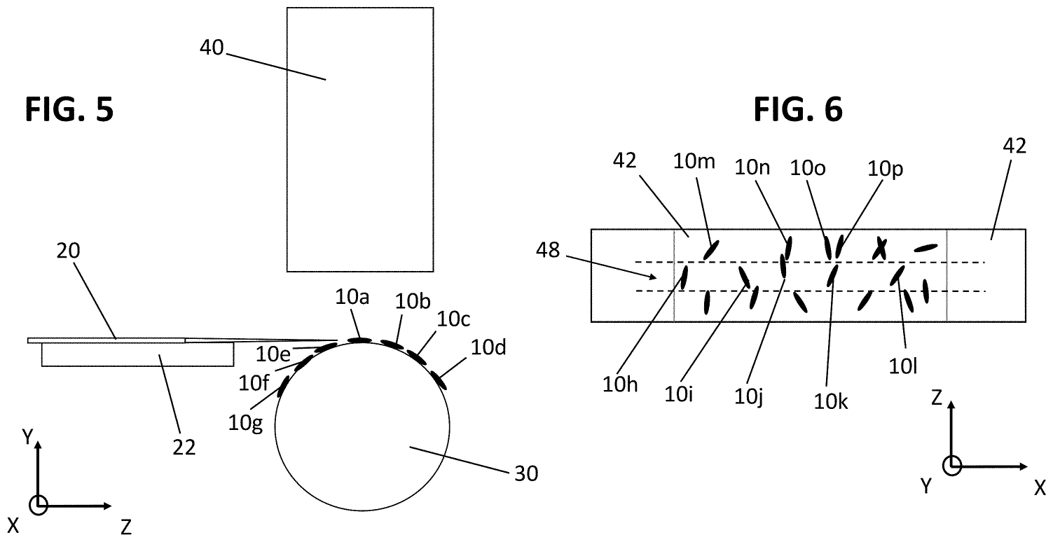

FIG. 4 shows a configuration of a micro-positioning system 5 for possible use in the microinjection of eggs such as insect eggs that addresses and potentially alleviates this problem of having some eggs block other eggs. In the system 5 shown, the eggs 10 are mounted on a shaft 30 having a curved surface. The eggs 10 are mounted on this shaft, e.g., as illustrate in FIG. 5. Referring again to the system 5 shown in FIG. 4, the pipette 20 is secured to a pipette holder 22 that is mounted on translation stages to move the pipette with respect to the shaft and consequently the eggs. The translation stages include first and second translations stage 24, 26 that may be configured to move in orthogonal directions. The first and second translation stages 24, 26 may comprise, for example, a lateral translation stage and a longitudinal translation stage, respectively. The lateral translation stage 24 may, for example, be configured to translate in a lateral direction such as in the x direction shown in FIG. 4 (e.g., parallel to the x axis), possibly in a direction left or right with respect to the eggs 10. The longitudinal translation stage 26 may, for example, be configured to translate in a longitudinal direction such as in the z direction shown in FIG. 1 (e.g., parallel to the z axis) in a direction toward the eggs 10. A third translation stage 32 may be configured to translate in a direction orthogonal to the first and second translation stages 24, 26. This third translation stage 32 may, for example, be configured to translate in the vertical direction (e.g., parallel to the y axis), possibly in a direction up or down with respect to the eggs 10. Accordingly, the lateral and vertical translation stages 24, 32 may be employed to align the pipette 20, and in particular, a tip 28 of the pipette with eggs 10 on the shaft 30. The longitudinal translation stage 26 can translate the pipette 20 forward so that the tip 28 of the pipette penetrates the egg 10 for injection.

In the example shown in FIG. 4, the first and second translation stages 24, 26 comprise electronically controllable translations stages. These first and second translation stages 24, 26 may be configured to receive electrical signals from electronics such as a processor that controls the movement of the translation stages. As described herein, in some implementations, artificial intelligence may be employed to control the translation stages 24, 26. For example, object detection and/or object segmentation may be applied to images of the eggs 10 to determine the location of the eggs and/or the end of the egg to move the translation stages 24, 26 so as to position the pipette tip 28 with respect to the egg. The translation stages 24, 26 may, for example, move the pipette 20 and the pipette 28 so as to inject the tip 28 of the pipette 20 into the egg 10, for example, into the end of the egg.

In various implementations, a camera 40 such as an overhead camera (shown in FIG. 5) that is configured to view the pipette from overhead the shaft 30 may be used to produced images of the eggs 10 on the shaft 30 and the pipette 20. Images from this camera may be used by the processor to determine the location of eggs and/or the pipette tip 38 using, for example, object detection, object segmentation or other artificial intelligence and/or computer vision approaches.

In the example shown in FIG. 4, the third translation stage 32 comprises a manual translation stage. The translation stage 32 comprises a handle 33 that can be rotated to cause translation of the translation stage. This handle 33 may comprise a vernier in various implementations. Accordingly, for certain designs, the height of the pipette 20 may be manually set. The user may, for example, view the pipette 20 from the side of the pipette to set the height of the pipette, for example, such that the pipette is at the same height of the egg 10. The user may also set the height to be just slightly above the egg 10 and use trial and error to determine that the pipette tip 28 is at the height of the egg. Other approaches are possible. For example, an image of the egg 10 and the pipette 20 can be observed to determine the height of the pipette such that the pipette and egg are in focus at the same time. A combination of such approaches may also be employed. In various implementations, a side viewing camera configured to view the pipette from a side may be used regardless of whether the third translation stage is manual or an electronically controlled translation stage that is controlled by a microprocessor. This camera may be oriented to view the eggs on the shaft as well and may be directed at an angle with respect to the length of the shaft and the length of the pipette.

In various implementations, however, the third translation stage 32 comprises an electronically controllable translations stage. This third translation stage 32 may be configured to receive electrical signals from electronics such as a processor that controls the movement of the translation stage. As described herein, in some implementations, artificial intelligence may be employed to control the translation stage 32. For example, object detection and/or object segmentation may be applied to images of the eggs 10 to determine the location of the eggs and/or the end of the egg to move the translation stages 24, 26 so as to position the pipette tip 28 with respect to the egg. The translation stages 24, 26 may, for example, move the pipette 20 and the pipette 28 so as to inject the tip 28 of the pipette 20 into the egg 10, for example, into the end of the egg.

FIG. 4 also illustrates the shaft 30 connected to a motor 34 using a coupler 36. For example, one end of the coupler 36 may be secured to a rotating shaft (not shown) of the motor 34 and the other end of the coupler may be secured to the shaft 30 on which the eggs 10 are supported. The shaft 30 is thereby configured to be rotated by the motor 34. The motor 30 may comprise, for example, a stepper motor in various designs, although the motor may comprise other types of motors such as a servo motor. An encoder may possibly be employed in certain cases. The eggs 10 are shown adhered to the shaft 10 with double-sided sticky tape 42. As is well known, double-sided sticky tape may comprise a layer or sheet of material such as plastic, likely optically transmissive or transparent plastic, with adhesive material on both sides of the layer or sheet. The adhesive material is also optically transmissive or transparent. Moreover, when applied to the shaft the double-sided sticky tape is optically transmissive and/or transparent.

The motor 34 is shown in FIG. 34 supported on an elevated platform 39 to attached to legs 44 that raised motor and shaft 30 to a suitable height such that the pipette can be positioned to a height the same as eggs 10 to the shaft. In some implementations, various components such as the translation stages 24, 26, 32 as well as possibly the legs 40 for the platform 38 may be mounted to a base 46 that potentially supports various components (e.g., motor, shaft, translation stages, camera(s) etc.) and secures the position of the various components with respect to each other.

FIG. 5 shows a cross-sectional view of the shaft 30 (orthogonal to the length of the shaft). A plurality of eggs 10a-10h, are depicted on the rotating shaft 30. In particular, the eggs 10a-10h are disposed on the surface of the shaft 30 an about a portion of the circumference of the shaft. As discussed above, the eggs 10a-10h may be on double-sided sticky tape adhered to the surface of the shaft 30.

The pipette 20 is also shown in FIG. 5 and is positioned to penetrate the end of the egg 10a on the top of the shaft 30. The shaft 30 can rotate to move different eggs 10 into position on the top of the shaft to provide unobstructed access of the pipette 20 to the egg. As shown in FIG. 5, for example, the shaft 30 is oriented such that the egg 10a shown on top of the shaft can receive the tip 38 of the pipette 20 without the pipette being intercepted by any other egg. No other eggs block access of the pipette 20 to the egg 10a shown on top of the shaft 30. To inject into the adjacent egg 10b the shaft 30 would rotate counter clockwise as shown in FIG. 5. This egg 10b would then be on top of the shaft 30 such that this egg 10a could receive the tip 38 of the pipette 20 without the pipette being intercepted by any other egg. No other eggs would block access of the pipette 20 to this egg 10b when the shaft 30 was rotated such that this egg was on top of the shaft. Similarly, the shaft could be rotated counter-clockwise again to allow the pipette to penetrate the next egg 10c. Conversely, the shaft 30 could be rotated clockwise to allow the pipette 20 to penetrate the egg 10e on the other side of the shaft 30. Similarly, the shaft 30 could be rotated any amount in either direction, clockwise or counter-clockwise, to access any of the eggs disposed around the circumference of the shaft.

FIG. 5 also shows a microscope camera 40 configured to images the pipette 20 and the eggs 10. The camera 40 illustrate comprise an overhead camera. This camera 40 is configured to image the top of the shaft 30 and the eggs on the top of the shaft. This camera may comprise a sensor such a detector array such as a CCD or CMOS detector or sensor array. The camera may include imaging optics to image the pipette, pipette tip, shaft, eggs or any combination thereof on the detector array. The optic may comprise microscope optics such as a microscope objective and may include additional optics. In various implementations the optics includes a work distance of at least a few millimeters. This work distance may, for example, be 5 mm, 8 mm, 10 mm, 12 mm, 15 mm, 20 mm, 25 mm, 30 mm, 35 mm, 40 mm, 50 mm, 60 mm, 70 mm or any range form by any of these values or larger or smaller.

FIG. 6 shows a top-view of the shaft 30 showing the eggs 10 on the shaft. As mentioned above, the eggs 10 may be adhered to the shaft 30 via double-sided sticky tape. FIG. 6 demarcates a zone or band 48, in this case on the top of the shaft 30, and shows a plurality of eggs within that zone. The system 5 may be configured to move the translation stages, e.g., the lateral and longitudinal translation stages 24, 26, to penetrate the eggs 10h-10l within this zone 48 and then rotate the shaft 30 with the motor 36 such that a new group of eggs 10m-10p is placed within this zone. The translation stages, e.g., the lateral and longitudinal translation stages 24, 26, can then be moved to sequentially penetrate the eggs 10m-10p within this band 48. In some cases, not all the eggs 10 in the band 48 may be penetrated, however.

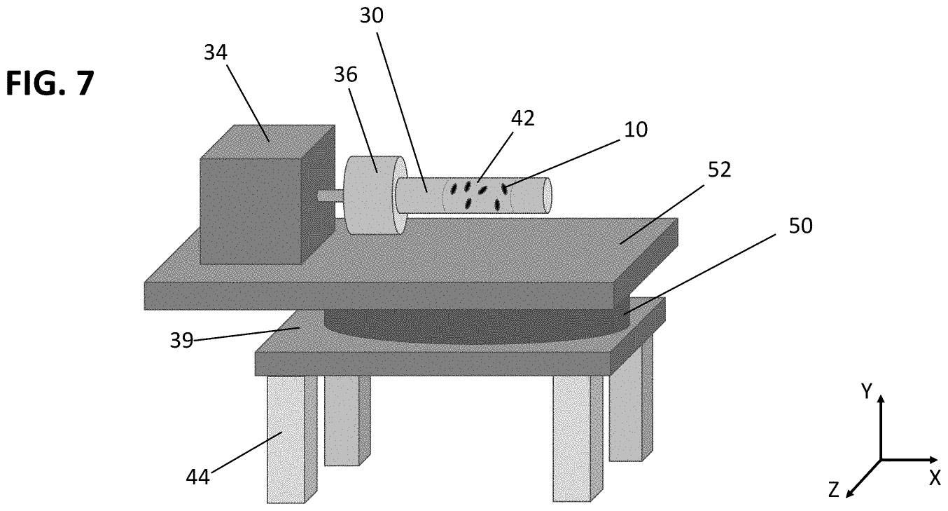

As discussed above, additional translation stages may be employed and may be configured to move the pipette 20 and/or the eggs 10. As illustrated in FIG. 7, in some implementations, a rotation stage 50 may be included in the system 5 to rotate the eggs 10 with respect to the pipette 20. In the example shown, the shaft 30 and motor 34 are mounted on a support 52 that is mounted on the rotation stage 50. The rotation stage 50 is supported on the platform 39 on the legs 44 shown in FIG. 7. The rotation stage 50 has an axis of rotation normal to length of the shaft 30 and normal to the plate 52, the platform 39 and the base 46 in the example shown. In some cases, therefore, this rotation axis may be normal to a plane formed by the x and z axis (e.g., the xz plane). In some such cases, the rotation axis may be directed in the vertical direction and may be parallel to the y-axis. In various implementations, the portion of the shaft where the eggs are mounted is above this axis of rotation.

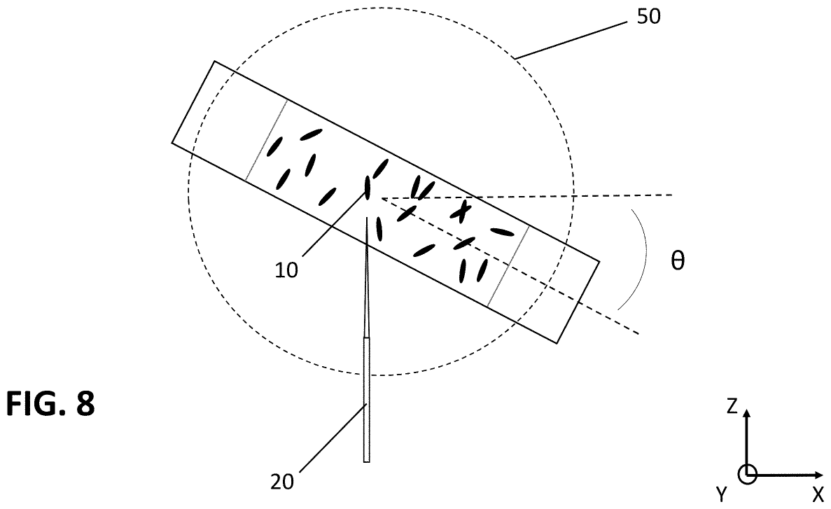

The rotation stage 50 may comprise an electronically controlled rotation stage that may be driven by a processor. As illustrated in FIG. 8, the rotation stage 50 may be configured to rotate the egg 10 and angle, θ, such that the length of the egg 10 is more aligned with the longitudinal direction. As a result, the pipette 20 may penetrate the egg 10 though an end of the egg as opposed to the side of the egg, which may be desirable in some case. Additionally, in some implementations, the rotations stage 50 may be configured to rotate at least 180° and possibly at least 360°. With such designs, the system 5 may be configured to rotate the rotation stage such that either end of the egg 10 may be penetrated with the pipette 20. In some cases, it may be beneficial to penetrate one of the ends of the egg 10 as opposed to the other end of the egg. Accordingly, in various implementations, electronics are configured to rotation the rotation stage 50, for example, so that the egg 10 is oriented in a particular orientation with respect to the pipette 20 (e.g., parallel to the longitudinal translation direction of the pipette and/or to inject into a particular side or end of the egg).

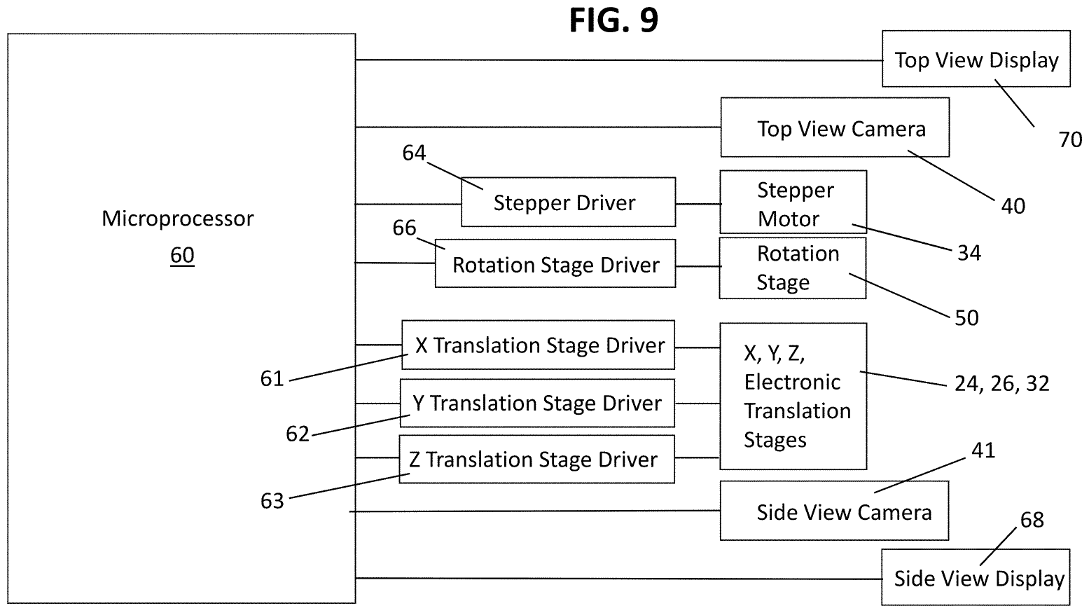

As discussed above, electronics provide signals to the one or more translation stages 24, 26, 32 to move the translation stages to such that the pipette 20 is injected into the egg 10. This electronics may comprise one or more microprocessors 60 such as shown in FIG. 9 and possibly additional electronics. FIG. 9 is a block diagram showing the microprocessor electrically 60 connected to a top camera 40 and an optional side camera 41 as well as drivers for x, y, and z electronically controlled translation stages 61, 62, 63 that are electrically connected to the respective translation stages 24, 26, 32. The microprocessor 60 is also shown electrically connected to a stepper motor driver 64 that is electrically connected to the motor 34 for rotating the shaft 30 as well as a driver 66 for driving the rotation stage 50.

In some implementations, any one of the drivers for the translation stages 61, 62, 63 may be integrated in the respective translation stage 24, 26, 32. Similarly, the driver 66 for the rotation stage 50 may be integrated in the rotation stage. Memory as well as a storage device may be in communication with the processor 60 and may store one or more software modules. One or more displays such as a top view display 70 and/or a side view display 68 or a single display the display both the top view and side view may be includes. Such top and side views may be provided by the overhead camera 40 and the side camera 41 respectively.

As discussed herein, images of the eggs and pipette can be capture by camera(s) 40, 41 and be processed by the electronics, e.g., the one or more processors 60. For example, computer vision and/or machine learning may be employed to detect eggs 10 in the images. Features of the detected egg in the image, such as the tip, center, side, may be identified. Edges of the egg may be detected. Bounding boxes and/or masks may be created based on the edges of the eggs. Data associated with the detected egg such as the location of the egg, the locations of features on the egg (e.g., the tip, center, centroid or any of these), the perimeter, the location of bounding box points or masks tracing the outline of the eggs or any combination of these may be determine and/or used.

Similarly, in various implementations, computer vision and/or machine learning may be employed to identify the pipette 20 in the images. Features of the detected pipette 20 in the image, such as the tip, center, side, may be identified. Edges of the pipette 20 may be detected. Bounding boxes and/or masks may be created based on the edges of the pipette 20. Data associated with the detected pipette 20 such as the location of the pipette, the locations of features on the pipette (e.g., the tip of the pipette, the center, centroid or any of these), the perimeter of the pipette, the location of bounding box points or masks tracing the outline of the pipette or any combination of these may be determine and/or used.

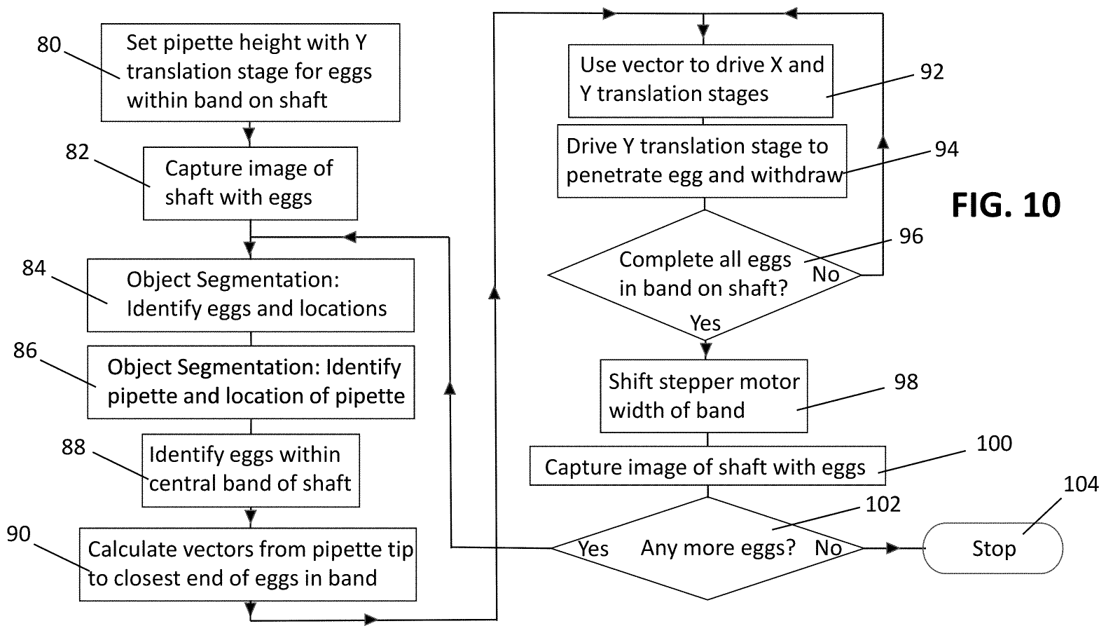

FIG. 10 describes an example method of using images from the camera 40 to determine the locations of the pipette tip and egg and move the translation stages to cause the tip to penetrate the eggs. In block on of the example, the pipette height is set with the vertical translation stage 32 for eggs 10 within the zone 48, for example, on the top of the shaft. This height might be set manually by adjusting the vertical translation stage 32, for example, while viewing a display (e.g., side display 68) provides an image of a side of the pipette 20 and shaft 30 obtained from the optional side camera 41. In block 82 an image of the shaft 30 is capture showing eggs on the shaft. In block 84 object segmentation is performed on the image to identify eggs in the image and their location and/or the location of features of the eggs is determined. In block 86, object segmentation is performed on the image to identify pipette in the image and the pipettes location and/or the location of features of the pipettes such as the location of the tip is determined. In block 88, the eggs in the central band or zone 48 of the shaft 30 are determined. In step 90, vectors from the determined location of the pipette tip and the location of the closest end of the eggs in the band is calculated. In step 92, the calculated vector is used to drive the lateral and longitudinal translation stages such that the pipette will move to the egg. An additional amount of longitudinal movement is provided for the longitudinal translation stage to cause the tip of the pipette to penetrate the egg as illustrated by block 92. In block 94, an amount of longitudinal movement in the opposite direction is provided for the longitudinal translation stage to cause the tip of the pipette to withdraw from the egg. At decision point 96, a determination is made whether all the eggs in the band have been penetrated by the egg, if not, the process goes back to block 92 where the vector for another egg in the zone 48 is used to drive the translation stage and the subsequent actions are completed until the decision point 96 is reached again. If yes, all the eggs in the band 48 have been penetrated, the motor 34 is caused to rotate the shaft such that a new group of eggs (comprising possibly only a single egg) are in the zone 48. An image of the shaft with the eggs thereon and the new group of egg in the zone is captured. If no eggs are in the band, the process stops otherwise the process goes back to block 84 where object detection and segmentation is performed on the eggs again.

The process shown in FIG. 10 can be varied in a wide range of ways. For example, the order of the steps may be different. A wider range of other variations are possible. For example, the vertical translation stage 32 may comprise an electronic translation stage in communication with the electronics such that height of the pipette is adjusted based on images obtained from a camera such as the optional side camera 41. Image detection and/or segmentation may be performed to on the eggs and pipette to determine how to move the vertical translation stage to set the height of the pipette based on the vertical location of the egg. Additionally, a wide range of methods for performing computer vision, object detection, object segmentation may be used. In some cases, the process is a machine learning process where training using images of eggs and images of pipettes are employed. Once trained, machine learning processes may be employed to make inferences and perform the object detection and/or object segmentations. Additional information regarding such computer vision and machine learning processes are provided below.

While the term pipette is employed herein this pipette 20 may comprises a micro pipette. This micro-pipette 20 may, for example, be 0.1 mm, 0.2 mm, 0.5 mm, 1 mm, 2 mm wide, or 3 mm wide at the widest parts or in any range formed by any of these values or larger or smaller. The tip 28 of the pipette or micro-pipette 20 may be much smaller, for example, be 1 micron, 5 microns, 10 microns, 20 microns, 50 microns, 100 microns, 120 microns, 150 microns, 200 microns, 250 microns, 300 microns, 400 microns, 500 microns or in any range formed by any of these values or larger or smaller.

The eggs 10 may be small. The eggs 10 may have a largest dimension, e.g., a length, of for example, be 1 micron, 5 microns, 10 microns, 20 microns, 50 microns, 100 microns, 120 microns, 150 microns, 200 microns, 250 microns, 300 microns, 400 microns, 500 microns, 600 microns, 700 microns, 800 microns, 900 microns, 1000 microns, 1200 microns, 1300 microns, 1400 microns, 1500 microns, 1600 microns, 1700 microns, 1800 microns, 1900 microns, 2 mm, 3 mm, 4 mm, 5 mm or in any range formed by any of these values or larger or smaller. The eggs 10 may have a smaller dimension, e.g., a width, of for example, be 1 micron, 5 microns, 10 microns, 20 microns, 50 microns, 100 microns, 120 microns, 150 microns, 200 microns, 250 microns, 300 microns, 400 microns, 500 microns, 600 microns, 700 microns, 800 microns, 900 microns, 1000 microns, 1200 microns, 1300 microns, 1400 microns, 1500 microns, 1600 microns, 1700 microns, 1800 microns, 1900 microns, 2 mm, 3 mm, 4 mm, 5 mm or in any range formed by any of these values or larger or smaller.

In various implementations, the eggs 10 are not spheres. In various case, the eggs 10 elongate having a length different than the width thereof. The eggs may for example have an aspect ratio length to width of 1.1, 1.2, 1.3, 1.4, 1.5, 2, 2.5, 3, 3.5, 4, 4.5, 5, 6, 7, 8 or any range formed by any of these values or larger or smaller. The eggs may also have complex shapes other than simple geometric shapes and/or may be irregularly shaped.

In various cases, the eggs comprise mosquito eggs but need not be so limited. The eggs may comprise other types of insect eggs. The eggs, however, need not be limited to insect eggs. The micro-positioning systems and microinjection system also need not be limited to eggs but can be used for other biological objects, for example, having such small size and/or having such shapes. However, the micro-positioning systems and microinjection system need not be limited to such biological objects as these.

While the pipette 20 is shown as being configured to penetrate eggs 10 on the top of the shaft 30, in different designs, the pipette can be configured to penetrate eggs on the bottom of the shaft or anywhere else on the shaft, for example, on a side of the shaft. One or more of the translations stages 24, 26, 32 may need to be oriented differently, for example, if the pipette penetrates eggs on the side of the shaft 30. Likewise, the translations stages 24, 26, 32 need not be oriented as they are shown in FIG. 4. For example, one or more translation stages 24, 26, 32 that translate in directions that are non-horizontal and non-vertical are possible, regardless whether the pipette 20 penetrates eggs 10 on the top of the shaft 30, the bottom of the shaft, on a side of the shaft, etc.

Additionally, the pipette holder may comprise different configurations. For example the pipette holder may comprise a rod or other elongate member and the pipette may be mounted the rod (e.g., on the top or bottom or side of the rod or other elongate member). The pipette may be mounted in an opening or hole in the holder such as an opening or hole in the face of the holder.

In some implementations, the pipette may be mounted in an groove on the holder such as on the top or bottom or side of the holder.

In some designs the holder is configured to support a plurality of pipettes. Moreover, in various implementations, the holder or a portion of the holder to which the pipettes are secured is configured to move, e.g., rotated, such that the pipette that was previously used to penetrate one or more eggs can be switched out for another pipette. In some designs, for example, the pipette holder is configured to rotate an array of pipettes such that the pipette that is used to penetrate the egg is switch out. A configuration similar to a revolver may be used. In some implementations, for example, the pipette below the holder is used to penetrate the egg. The holder can be configure to rotate an array of pipettes arrange in a ring so that another pipette is located the location below the holder to penetrate the egg. Such a configuration may be useful to address potential clocking of pipettes after penetration into the egg(s).

### Computer Vision to Detect Objects in Ambient Environment

As discussed above, the microinjection system may be configured to detect objects such as eggs and pipettes. The detection may be accomplished using a variety of techniques, including sensors such as cameras as discussed herein.

In some embodiments, objects present in images captured by the one or more cameras may be detected using computer vision techniques. For example, as disclosed herein, the display system's microscope camera 40 (or side view camera 41) may be configured to image the eggs and the pipette and the microinjection system may be configured to perform image analysis on the images to determine the presence of objects such as eggs and the pipette in the images. The microinjection system may analyze the images acquired by the microscope camera to perform scene reconstruction, event detection, video tracking, object recognition, object segmentation, object pose estimation, learning, indexing, motion estimation, or image restoration, etc. As other examples, the micro-positioning and/or microinjection system may be configured to perform object recognition and/or segmentation to determine the presence and location of eggs and/or one or more pipettes in the camera's field of view. One or more computer vision algorithms may be used to perform these tasks. Non-limiting examples of computer vision algorithms include: Scale-invariant feature transform (SIFT), speeded up robust features (SURF), oriented FAST and rotated BRIEF (ORB), binary robust invariant scalable keypoints (BRISK), fast retina keypoint (FREAK), Viola-Jones algorithm, Eigenfaces approach, Lucas-Kanade algorithm, Horn-Schunk algorithm, Mean-shift algorithm, visual simultaneous location and mapping (vSLAM) techniques, a sequential Bayesian estimator (e.g., Kalman filter, extended Kalman filter, etc.), bundle adjustment, Adaptive thresholding (and other thresholding techniques), Iterative Closest Point (ICP), Semi Global Matching (SGM), Semi Global Block Matching (SGBM), Feature Point Histograms, various machine learning algorithms (such as e.g., support vector machine, k-nearest neighbors algorithm, Naive Bayes, neural network (including convolutional or deep neural networks), or other supervised/unsupervised models, etc.), and so forth.

### Machine Learning

A variety of machine learning algorithms may be used to learn to identify the presence of objects in the image. Once trained, the machine learning algorithms may be stored by the system. Some examples of machine learning algorithms may include supervised or non-supervised machine learning algorithms, including regression algorithms (such as, for example, Ordinary Least Squares Regression), instance-based algorithms (such as, for example, Learning Vector Quantization), decision tree algorithms (such as, for example, classification and regression trees), Bayesian algorithms (such as, for example, Naive Bayes), clustering algorithms (such as, for example, k-means clustering), association rule learning algorithms (such as, for example, a-priori algorithms), artificial neural network algorithms (such as, for example, Perceptron), deep learning algorithms (such as, for example, Deep Boltzmann Machine, or deep neural network), dimensionality reduction algorithms (such as, for example, Principal Component Analysis), ensemble algorithms (such as, for example, Stacked Generalization), and/or other machine learning algorithms. In some embodiments, individual models may be customized for individual data sets. For example, the system may generate or store a base model. The base model may be used as a starting point to generate additional models specific to a data type (e.g., a particular user), a data set (e.g., a set of additional images obtained), conditional situations, or other variations. In some embodiments, the system may be configured to utilize a plurality of techniques to generate models for analysis of the aggregated data. Other techniques may include using pre-defined thresholds or data values.

The criteria for detecting an object may include one or more threshold conditions. If the analysis of the data acquired by the camera(s) indicates that a threshold condition is passed, the system may provide a signal indicating the detection the presence of the object in the image. The threshold condition may involve a quantitative and/or qualitative measure. For example, the threshold condition may include a score or a percentage associated with the likelihood of the object being present. The system may compare the score with the threshold score. If the score is higher than the threshold level, the system may detect the presence of the object. In some other embodiments, the system may signal the presence of the object if the score is lower than the threshold.

In some embodiments, the threshold conditions, the machine learning algorithms, or the computer vision algorithms may be specialized for a specific context.

It will be appreciated that each of the processes, methods, and algorithms described herein and/or depicted in the figures may be embodied in, and fully or partially automated by, code modules executed by one or more physical computing systems, hardware computer processors, application-specific circuitry, and/or electronic hardware configured to execute specific and particular computer instructions. For example, computing systems may include general purpose computers (e.g., servers) programmed with specific computer instructions or special purpose computers, special purpose circuitry, and so forth. A code module may be compiled and linked into an executable program, installed in a dynamic link library, or may be written in an interpreted programming language. In some embodiments, particular operations and methods may be performed by circuitry that is specific to a given function.

Further, certain embodiments of the functionality of the present disclosure are sufficiently mathematically, computationally, or technically complex that application-specific hardware or one or more physical computing devices (utilizing appropriate specialized executable instructions) may be necessary to perform the functionality, for example, due to the volume or complexity of the calculations involved or to provide results substantially in real-time. For example, a video may include many frames, with each frame having millions of pixels, and specifically programmed computer hardware is necessary to process the video data to provide a desired image processing task or application in a commercially reasonable amount of time.

Code modules or any type of data may be stored on any type of non-transitory computer-readable medium, such as physical computer storage including hard drives, solid state memory, random access memory (RAM), read only memory (ROM), optical disc, volatile or non-volatile storage, combinations of the same and/or the like. In some embodiments, the non-transitory computer-readable medium may be part of one or more of the local processing and data module, the remote processing module, and remote data repository. The methods and modules (or data) may also be transmitted as generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). The results of the disclosed processes or process steps may be stored, persistently or otherwise, in any type of non-transitory, tangible computer storage or may be communicated via a computer-readable transmission medium.

Any processes, blocks, states, steps, or functionalities in flow diagrams described herein and/or depicted in the attached figures should be understood as potentially representing code modules, segments, or portions of code which include one or more executable instructions for implementing specific functions (e.g., logical or arithmetical) or steps in the process. The various processes, blocks, states, steps, or functionalities may be combined, rearranged, added to, deleted from, modified, or otherwise changed from the illustrative examples provided herein. In some embodiments, additional or different computing systems or code modules may perform some or all of the functionalities described herein. The methods and processes described herein are also not limited to any particular sequence, and the blocks, steps, or states relating thereto may be performed in other sequences that are appropriate, for example, in serial, in parallel, or in some other manner. Tasks or events may be added to or removed from the disclosed example embodiments. Moreover, the separation of various system components in the embodiments described herein is for illustrative purposes and should not be understood as requiring such separation in all embodiments. It should be understood that the described program components, methods, and systems may generally be integrated together in a single computer product or packaged into multiple computer products.

### EXAMPLES

Some additional nonlimiting examples of embodiments discussed above are provided below. These should not be read as limiting the breadth of the disclosure in any way.

Example 1: A micro-positioning system configured to insert a pipette into a plurality of eggs, said micro-positioning system comprising:
a pipette holder configured to secure said pipette to said pipette holder;
a shaft for mounting a plurality of eggs, said pipette holder movable in lateral, longitudinal, and vertical directions with respect to said shaft to position said pipette with respect to said eggs;
at least one electronically controlled lateral translation stage configured to translate in the lateral direction so as to move said pipette holder laterally with respect to said shaft;
at least one electronically controlled longitudinal translation stage configured to translate in the longitudinal direction so as to move said pipette holder longitudinally with respect to said shaft;
at least one vertical translation stage configured to translate in the vertical direction so as to move said pipette holder vertically with respect to said shaft;
a motor connected to said shaft to rotate said shaft so as to provide said pipette to access to different eggs mounted on said shaft; and
electronics configured to drive at least said lateral and longitudinal translation stages and said motor to position said pipette with respect to said egg.

Example 2: The microinjection system of Example 1, wherein said pipette holder comprises a hole or groove configured to receive said pipette.

Example 3: The microinjection system of Example 1 or 2, wherein said pipette is configured to be supported on said electronically controlled lateral translation stage such that said pipette is moved laterally.

Example 4: The microinjection system of any of the examples above, wherein said pipette is configured to be supported on said electronically controlled longitudinal translation stage such that said pipette is moved longitudinally.

Example 5: The microinjection system of any of the examples above, wherein said pipette is configured to be supported on said vertical translation stage such that said pipette is moved vertically.

Example 6: The microinjection system of any of the examples above, wherein said shaft is configured to be supported on said electronically controlled lateral translation stage such that said shaft is moved laterally.

Example 7: The microinjection system of any of the examples above, wherein said shaft is configured to be supported on said electronically controlled longitudinal translation stage such that said shaft is moved longitudinally.

Example 8: The microinjection system of any of the examples above, wherein said shaft is configured to be supported on said vertical translation stage such that said pipette is moved vertically.

Example 9: The microinjection system of any of the examples above, wherein said vertical translation stage comprises an electronically controlled vertical translation stage configured to translate in the vertical direction so as to move said pipette holder vertically with respect to said shaft.

Example 10: The microinjection system of any of the examples above, wherein said motor comprises a stepper motor or a servo motor.

Example 11: The microinjection system of any of the examples above, further comprising at least one camera including microscope optics configured to images said pipette and said eggs.

Example 12: The microinjection system of Example 11, wherein said at least one camera comprises a camera located vertically above said shaft to top of said pipette.

Example 13: The microinjection system of Example 11 or 12, wherein said at least one camera comprises a side camera configured to view a side of said pipette.

Example 14: The microinjection system of any of Examples 11-13, wherein said electronics are configured to received images from said at least one camera of said eggs and said pipette and to drive at least said lateral and longitudinal translation stages and said motor to position said pipette with respect to said egg such that said pipette penetrates said egg based on said images from said camera.

Example 15: The microinjection system of any of the examples above, further comprising at least one electronically controlled rotation stage configured to rotate said shaft with respect to said pipette holder.

Example 16: The microinjection system of Example 13, wherein said one electronically controlled rotation stage is configured to rotate about an axis orthogonal the length of the shaft.

Example 17: The microinjection system of Example 13 or 14, wherein said one electronically controlled rotation stage is configured to rotate in a plane parallel to said longitudinal and lateral directions.

Example 18: The microinjection system of Example 13 or 14, wherein electronics are configured to control said electronically controlled rotation stage.

Example 19: The microinjection system of any of the claims above, wherein said pipette holder is configured to hold a plurality of pipettes.

Example 20: The microinjection system of Example 12, wherein said pipette holder can move to change the pipette among the plurality of pipettes that is injected into the egg.

Example 21: The microinjection system of Example 13, wherein said pipette holder can rotate to change the pipette that among the plurality of pipettes is injected into the egg.

Example 22: A micro-positioning system configured to insert a pipette into a plurality of elongate eggs, said elongate eggs having ends and sides, said ends separated by a length, said elongate eggs having ends separated by a length and having sides separated by a wide, said length longer than said width, said micro-positioning system comprising:
a pipette holder configured to secure said pipette to said pipette holder;
a platform for mounting a plurality of elongate eggs, said pipette holder movable in lateral, longitudinal, and vertical directions with respect to said platform to position said pipette with respect to said elongate eggs;
at least one electronically controlled lateral translation stage configured to translate in the lateral direction so as to move said pipette holder laterally with respect to said platform;
at least one electronically controlled longitudinal translation stage configured to translate in the longitudinal direction so as to move said pipette holder longitudinally with respect to said platform;
at least one vertical translation stage configured to translate in the vertical direction so as to move said pipette holder vertically with respect to said platform;
at least one camera including microscope optics configured to images said pipette and said elongate eggs, and
electronics configured to drive at least said lateral and longitudinal translation stages to position said pipette with respect to said elongate egg. said electronics configured to received images from said at least one camera of said eggs and said pipette and to drive at least said lateral and longitudinal translation stages to position said pipette with respect to said end of said egg such that said pipette penetrates said elongate egg based on said images from said camera.

Example 23: The microinjection system of Example 22, wherein said platform comprises a shaft mounted on a motor.

Example 24: The microinjection system of Example 22 or 23, wherein said electronics are configured to drive at least said lateral and longitudinal translation stages to position said pipette with respect to one of said ends of said elongate egg such that said pipette penetrates said egg through said end of said elongate egg based on said images from said camera.

Example 25: The microinjection system of any of Examples 22-24, wherein said electronics are configured to identify said eggs in said images.

Example 26: The microinjection system of any of Examples 22-25, wherein said electronics are configured to identify said pipette in said images.

Example 27: The microinjection system of any of Examples 22-26, wherein said electronics are configured to drive said lateral and translation stages by distances such that said pipette penetrates said elongate egg through the end of said egg.

Example 28: The microinjection system of any of Examples 22-26, wherein said electronics are configured to drive said lateral and translation stages by distances such that said pipette penetrates said elongate egg through a side of said egg.

Example 29: A method of inserting a pipette into a plurality of elongate eggs, said elongate eggs having ends and sides, said ends separated by a length, said elongate eggs having ends separated by a length and having sides separated by a wide, said length longer than said width, said method comprising:
securing a pipette to a pipette holder;
mounting a plurality of elongate eggs on a platform,
moving said pipette in lateral, longitudinal, and vertical directions with respect to said platform to position said pipette with respect to said elongate eggs using:
   at least one electronically controlled lateral translation stage configured to translate in the lateral direction so as to move said pipette holder laterally with respect to said platform;
   at least one electronically controlled longitudinal translation stage configured to translate in the longitudinal direction so as to move said pipette holder longitudinally with respect to said platform;
   at least one vertical translation stage configured to translate in the vertical direction so as to move said pipette holder vertically with respect to said platform; and
   at least one camera including microscope optics configured to images said pipette and said elongate eggs,
sending signals from said camera to electronics to provide images to said electronics; and
sending signals from said electronics to drive at least said lateral and longitudinal translation stages to position said pipette with respect to said elongate egg such that said pipette penetrates one end of said elongate egg, said signals from said electronics being based on said images from said camera.

Example 30: The method of Example 29, further comprising performing object detection of said object and said pipette to determine said signal sent to said lateral and longitudinal translation stages.

Example 31: The method of any of Examples 29 or 30, further comprising performing object segmentation of said object and said pipette and moving said pipette with respect to said elongate eggs based on information obtained from said object segmentation.

Example 32: The method of any of Examples 29-31, wherein said eggs are moved to cause said pipette to be injected into said eggs.

Example 33: The method of any of Examples 29-31, wherein said pipette is moved to cause said pipette to be injected into said eggs.

Example 34: A method of inserting a pipette into a plurality of elongate eggs, said elongate eggs having ends and sides, said ends separated by a length, said elongate eggs having ends separated by a length and having sides separated by a wide, said length longer than said width, said method comprising:
securing a pipette to a pipette holder;
mounting a plurality of elongate eggs on a platform,
obtaining images of said eggs and said pipette;
moving said pipette in lateral, longitudinal, and vertical directions with respect to said platform to position said pipette with respect to said elongate eggs based on information obtained from said object detection of at least one eggs and said pipette so as inject said pipette into an end of said elongate egg.

Example 35: The method of Example 34, further comprising object segmentations of said object and said pipette and moving said pipette with respect to said elongate eggs based on information obtained from said object segmentation.

Example 36: The method of any of Examples 34 or 35, wherein said eggs are moved to cause said pipette to be injected into said eggs.

Example 37: The method of any of Examples 34 or 35, wherein said pipette is moved to cause said pipette to be injected into said eggs.

### Other Considerations

Each of the processes, methods, and algorithms described herein and/or depicted in the attached figures may be embodied in, and fully or partially automated by, code modules executed by one or more physical computing systems, hardware computer processors, application-specific circuitry, and/or electronic hardware configured to execute specific and particular computer instructions. For example, computing systems can include general purpose computers (e.g., servers) programmed with specific computer instructions or special purpose computers, special purpose circuitry, and so forth. A code module may be compiled and linked into an executable program, installed in a dynamic link library, or may be written in an interpreted programming language. In some implementations, particular operations and methods may be performed by circuitry that is specific to a given function.

Further, certain implementations of the functionality of the present disclosure are sufficiently mathematically, computationally, or technically complex that application-specific hardware or one or more physical computing devices (utilizing appropriate specialized executable instructions) may be necessary to perform the functionality, for example, due to the volume or complexity of the calculations involved or to provide results substantially in real-time. For example, animations or video may include many frames, with each frame having millions of pixels, and specifically programmed computer hardware is necessary to process the video data to provide a desired image processing task or application in a commercially reasonable amount of time.

Code modules or any type of data may be stored on any type of non-transitory computer-readable medium, such as physical computer storage including hard drives, solid state memory, random access memory (RAM), read only memory (ROM), optical disc, volatile or non-volatile storage, combinations of the same and/or the like. The methods and modules (or data) may also be transmitted as generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). The results of the disclosed processes or process steps may be stored, persistently or otherwise, in any type of non-transitory, tangible computer storage or may be communicated via a computer-readable transmission medium.

Any processes, blocks, states, steps, or functionalities in flow diagrams described herein and/or depicted in the attached figures should be understood as potentially representing code modules, segments, or portions of code which include one or more executable instructions for implementing specific functions (e.g., logical or arithmetical) or steps in the process. The various processes, blocks, states, steps, or functionalities can be combined, rearranged, added to, deleted from, modified, or otherwise changed from the illustrative examples provided herein. In some embodiments, additional or different computing systems or code modules may perform some or all of the functionalities described herein. The methods and processes described herein are also not limited to any particular sequence, and the blocks, steps, or states relating thereto can be performed in other sequences that are appropriate, for example, in serial, in parallel, or in some other manner. Tasks or events may be added to or removed from the disclosed example embodiments. Moreover, the separation of various system components in the implementations described herein is for illustrative purposes and should not be understood as requiring such separation in all implementations. It should be understood that the described program components, methods, and systems can generally be integrated together in a single computer product or packaged into multiple computer products. Many implementation variations are possible.

The processes, methods, and systems may be implemented in a network (or distributed) computing environment. Network environments include enterprise-wide computer networks, intranets, local area networks (LAN), wide area networks (WAN), personal area networks (PAN), cloud computing networks, crowd-sourced computing networks, the Internet, and the World Wide Web. The network may be a wired or a wireless network or any other type of communication network.

The systems and methods of the disclosure each have several innovative aspects, no single one of which is solely responsible or required for the desirable attributes disclosed herein. The various features and processes described above may be used independently of one another, or may be combined in various ways. All possible combinations and subcombinations are intended to fall within the scope of this disclosure. Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. No single feature or group of features is necessary or indispensable to each and every embodiment.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted can be incorporated in the example methods and processes that are schematically illustrated. For example, one or more additional operations can be performed before, after, simultaneously, or between any of the illustrated operations. Additionally, the operations may be rearranged or reordered in other implementations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

1. A micro-positioning system configured to insert a pipette into a plurality of eggs, said micro-positioning system comprising:
a pipette holder configured to secure said pipette to said pipette holder;
a shaft for mounting a plurality of eggs, said pipette holder movable in lateral, longitudinal, and vertical directions with respect to said shaft to position said pipette with respect to said eggs;
at least one electronically controlled lateral translation stage configured to translate in the lateral direction so as to move said pipette holder laterally with respect to said shaft;
at least one electronically controlled longitudinal translation stage configured to translate in the longitudinal direction so as to move said pipette holder longitudinally with respect to said shaft;
at least one vertical translation stage configured to translate in the vertical direction so as to move said pipette holder vertically with respect to said shaft;
a motor connected to said shaft to rotate said shaft so as to provide said pipette access to different eggs mounted on said shaft; and
electronics configured to drive at least said lateral and longitudinal translation stages and said motor to position said pipette with respect to said egg.

2. The microinjection system of claim 1, wherein said pipette holder comprises a hole or groove configured to receive said pipette.

3. The microinjection system of claim 1, wherein said pipette is configured to be supported on said electronically controlled lateral translation stage such that said pipette is moved laterally.

4. The microinjection system of claim 1, wherein said pipette is configured to be supported on said electronically controlled longitudinal translation stage such that said pipette is moved longitudinally.

5. The microinjection system of claim 1, wherein said pipette is configured to be supported on said vertical translation stage such that said pipette is moved vertically.

6. (canceled)

7. (canceled)

8. (canceled)

9. The microinjection system of claim 1, wherein said vertical translation stage comprises an electronically controlled vertical translation stage configured to translate in the vertical direction so as to move said pipette holder vertically with respect to said shaft.

10. The microinjection system of claim 1, wherein said motor comprises a stepper motor or a servo motor.

11. The microinjection system of claim 1, further comprising at least one camera including microscope optics configured to image said pipette and said eggs.

12. The microinjection system of claim 11, wherein said at least one camera comprises a camera located vertically above said shaft to view said top of said pipette.

13. The microinjection system of claim 11, wherein said at least one camera comprises a side camera configured to view a side of said pipette.

14. The microinjection system of claim 11, wherein said electronics are configured to receive images from said at least one camera of said eggs and said pipette and to drive at least said lateral and longitudinal translation stages and said motor to position said pipette with respect to said egg such that said pipette penetrates said egg based on at least one of said images from said camera.

15. The microinjection system of claim 1, further comprising at least one electronically controlled rotation stage configured to rotate said shaft with respect to said pipette holder.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
